(19) United States
(12) Patent Application Publication
(10) Pub. No.: US 2022/0218743 A1
(43) Pub. Date: Jul. 14, 2022

(54) SILVER FLUORIDE SOLUTIONS FOR DENTAL COMPOSITIONS

(71) Applicant: SDI Limited, Bayswater, Victoria (AU)

(72) Inventors: Paul FARRAR, Bayswater (AU); Amanda TARGETT, Bayswater (AU); Mingdeng LUO, Bayswater (AU)

(21) Appl. No.: 17/615,093

(22) PCT Filed: Feb. 7, 2019
(86) PCT No.: PCT/AU19/00014

§ 371 Date: Nov. 29, 2021

(30) Foreign Application Priority Data
Feb. 12, 2018 (AU) 2018900432

(51) Int. Cl. A61K 33/38; A61K 8/21; A61Q 11/00; A61P 1/02; A61P 31/04

(52) U.S. Cl. CPC A61K 33/38; A61K 8/21; A61K 2800/52; A61P 1/02; A61P 31/04; A61Q 11/00

(57) ABSTRACT

The present invention relates to an aqueous Silver Fluoride (AgF) containing solutions with pH 4.0-5.3.

### FIELD OF THE INVENTION

The present invention relates to a silver fluoride solutions for dental compositions.

### BACKGROUND

Silver fluoride containing products have been known for many years to provide therapeutic benefits as a treatment of dental hard tissues. The solutions have generally been presented as aqueous solutions of Silver Fluoride (AgF), Silver Nitrate (AgNO₃) or Silver Diammine Fluoride (SDF). Specifically, these products are known to provide protection to affected teeth by killing the bacteria associated with dental caries and then promoting the remineralization of the demineralized collagen. In addition, these products can also be used to reduce tooth sensitivity by occluding the fluid movement through the dentinal tubules.

The silver fluoride containing products have the common drawback of changing the colour of the carious affected tooth to a permanent black shade due to oxidation of the silver ions. The oral soft tissues (gums, cheeks, lips, etc.) will also become blackened if contacted with the silver fluoride containing solution although this is only temporary and will wear off over the proceeding days.

One method of alleviating the black staining is to use the silver fluoride containing products with a subsequent iodide-salt-containing solution which, when applied immediately after the silver fluoride containing solution, reacts readily with free silver ions to form water-insoluble, cream-coloured silver iodide salts. After this treatment method, the carious lesion will now appear a brownish tinge (rather than black) and other dentinal tissues (non-carious tooth, gingiva, etc.) will appear unchanged as the excess silver iodide salts are rinsed away.

The silver fluoride containing solutions often exhibit poor stability upon storage due to the following reasons.
(1) AgF solutions tend to precipitate out of solution upon storage. The precipitate tends to form as a layer on the internal surface and/or settles to the bottom of the storage container (E.g. bottle). This reduction in the concentration of soluble ions thereby diminishes the effectiveness of the dental treatment.
(2) The AgF solutions can be stabilized by the addition of ammonia to form silver diammine fluoride (SDF). The ammonia combines with silver ions to produce a complex ion called the diamminesilver ion, [Ag(NH₃)₂]⁺. This is a reversible reaction, but the complex is very stable. Ammonia-containing compositions are, however, difficult to store due to the high volatility of ammonia. Bottles and containers readily lose ammonia upon storage, particularly at high temperature, causing the remaining AgF solution to become unstable (resulting in precipitation).

The ammonia used to produce the stable silver complex causes the pH to rise to hazardous levels within many commercial products (pH>10). As well as being difficult to store for long periods (due to ammonia volatility) the more stable SDF solution, unfortunately, is very irritating to oral soft tissues and can result in chemical burns if left in contact for extended periods.

It would be advantageous to develop a storage stable silver fluoride containing solution that exhibited caries arrest and desensitization of teeth whilst being gentle to the oral soft tissues. A suitable system is be based on substantially aqueous silver fluoride solutions that are stabilized in the region of pH 4 to 5.3.

The stabilized silver fluoride solution of the present invention is also suitable for use with iodide-salt-containing solutions to reduce staining as described above.

To achieve a reduction in the pH of an SDF solution, ammonia was omitted, rendering an aqueous silver fluoride solution. A freshly made aqueous solution of 38% w/v silver fluoride typically has a pH of 6.2-6.4, which upon storage is not stable and silver begins to precipitate out of solution. By adjusting the pH of the solution with an acid to a pH of 4.0-5.3, stability of the solution is achieved. pH values less than 4.0 are deemed unsuitable as this may cause chemical burns and irritation due to the corrosive nature of acidic solutions. pH values above 5.3 prove to be unstable for long term storage, with precipitation of silver from the solution. Nitric acid is a preferred compound for use as the acidic pH regulator, however other organic or inorganic acids that form soluble silver salts can also be used. This avoids precipitation of insoluble silver salts which may form as reaction products.

### EXAMPLE

Three aqueous 38% (w/v) silver fluoride products were prepared, with each solution exhibiting a different pH. The products were tested for the performance attributes detailed in Table 1.

TABLE 1

| | | Comparative | Comparative |
|---|---|---|---|
| | Example 1 | Example 1 | Example 2 |
| Product | 38% AgF w/v | 38% AgF w/v | 38% AgF w/v |
| | (stabilized with | (not stabilized) | (stabilized with |
| | nitrate) | | ammonia) |
| pH | 5.1 | 6.3 | 13.0 |
| Appearance | Clear colour- | Clear colour- | Clear colour- |
| (1 year @ 23° C.) | less liquid, no | less liquid, | less liquid, no |
| | (silver) sedi- | (silver) sedi- | (silver) sedi- |
| | ment | ment present | ment |
| AgF content in | 37.6% w/v | 9.6% w/v | 37.7% w/v |
| solution | | | |
| (1 year @ 23° C.) | | | |
| * Hydraulic | 87.2 | 86.2 | 89.4 |
| conductance (Lp %) | | | |
| (AgF + KI) | | | |

* Hydraulic conductance (Lp %) is used to determine the reduction of fluid movement through dentinal tubules (0%—No occlusion, 100%—complete occlusion, no fluid movement). Fluid movement through dentinal tubules can be the cause of tooth hypersensitivity, therefore Lp % represents the desensitizing effect. The application of a second step (Potassium Iodide solution) was applied to all example products to test whether the silver iodide precipitate will form to occlude dentin tubules with each of the different silver fluoride solutions.

### CONCLUSION

Ex 1: Good stability upon storage, good tubule occlusion and biologically compatible pH (no soft-tissue irritation)
Comp Ex 1: Poor stability upon storage, good tubule occlusion and biologically compatible pH (no soft-tissue irritation).
Comp Ex 2: Good Stability upon storage, good tubule occlusion, biologically incompatible pH (soft-tissue irritation.

Modifications and variations as would be apparent to a skilled addressee are deemed to be within the scope of the present invention.

1. An aqueous Silver Fluoride (AgF) containing solution with pH 4.0-5.3, wherein the composition is stabilized with an acid.

2. An aqueous Silver Fluoride solution as defined in claim 1, wherein the acid is nitric acid.

3. An aqueous Silver Fluoride solution as defined in claim 1, wherein the quantity of Silver Fluoride in the aqueous Silver Fluoride solution is about and up to 50% w/v.

4. An aqueous Silver Fluoride solution as defined in claim 2, wherein the quantity of Silver Fluoride in the aqueous Silver Fluoride solution is about and up to 50% w/v.
